(19) United States
(12) Patent Application Publication
(10) Pub. No.: US 2024/0325442 A1
(43) Pub. Date: Oct. 3, 2024

(54) CAR VECTOR EXPRESSING IMMUNE REGULATORY FACTOR AND APPLICATION THEREOF

(71) Applicant: West China Hospital of Sichuan University, Chengdu (CN)

(72) Inventors: Hanshuo YANG, Chengdu (CN); Qiqi LI, Chengdu (CN)

(73) Assignee: West China Hospital of Sichuan University, Chengdu (CN)

(21) Appl. No.: 18/288,782

(22) PCT Filed: Apr. 29, 2022
(86) PCT No.: PCT/CN2022/090106

§ 371 Date: Oct. 27, 2023

(30) Foreign Application Priority Data
Apr. 30, 2021 (CN) 202110481979.9

(51) Int. Cl. A61K 35/17; A61K 9/00; A61K 39/00; A61P 35/00; C07K 14/535; C07K 14/725; C12N 5/0783; C12N 15/86

(52) U.S. Cl. CPC A61K 35/17; A61K 9/0019; A61K 39/4611; A61K 39/464406; A61P 35/00; C07K 14/535; C07K 14/7051; C12N 5/0636; C12N 15/86; A61K 2239/15; A61K 2239/21; A61K 2239/22; A61K 2239/57; A61K 2239/59; C07K 2319/03; C12N 2740/15043

(57) ABSTRACT

The present invention relates to the field of bioengineering, and in particular to a chimeric antigen receptor (CAR) expression vector and use thereof. The CAR expression vector comprising a polynucleotide encoding a CAR and full length or fragment of a polynucleotide encoding a granulocyte-macrophage colony-stimulating factor (GM-CSF). The CAR-GM-T cell constructed by the present invention is capable of expressing high-level GM-CSF, not only directly enhancing killing activity of the CAR-GM-T cell per se but also facilitating the infiltration of the CAR-GM-T cell into a solid tumor. The CAR-GM-T cell has a stronger immune modulation function compared to the conventional CAR-T cell and systematically triggers the endogenous anti-tumor immune response, thereby achieving superior therapeutic efficacy against a solid tumor.

The present application claims priority from Chinese invention patent application CN2021104819799 “CAR vector expressing immune regulatory factor and application thereof” filed on Apr. 30, 2021, which is incorporated by reference in its entirety.

### TECHNICAL FIELD

The present invention relates to the field of bioengineering, and in particular to a CAR expression vector and use thereof.

### BACKGROUND

Chimeric antigen receptor T cells (CAR-T) have shown promising clinical efficacy in the treatment of hematologic malignancies such as B-cell acute lymphoblastic leukemia.

However, their efficacy against solid tumors has been limited. The CAR structure comprises three components: an extracellular domain for tumor antigen recognition, a transmembrane region for anchoring the CAR structure, and an intracellular signaling domain for T-cell activation and signal transduction. CAR-T-induced killing of tumor cells relies on the extracellular single-chain variable region binding specifically to tumor-associated antigens and initiating downstream signaling through the intracellular signaling domain, resulting in CAR-T cell activation, proliferation, and cytotoxicity by releasing cytokines such as IFN-γ.

Nonetheless, current conventional CAR-T therapy shows limited efficacy when facing solid tumors.

The first-generation CAR structure contains only 3 CD3ξ-derived ITAM sequences or 2 FcRγ-derived ITAM sequences in the intracellular domain which can only provide the first signal for T cell activation with limited capacity to secrete IL-2 and weak proliferation capacity, leading to unsatisfactory results in clinical trials. In the second-generation CAR structure, the intracellular signaling sequence is in tandem with a co-stimulatory molecule signaling domain, leading to enhanced proliferation and killing capacity compared to the first-generation CAR structure. The third-generation CAR structure includes more co-stimulatory domains in the intracellular signaling domain. However, studies have shown that its killing activity isn't significantly enhanced, and it causes more side effects. The fourth-generation CAR structure incorporates a new functional component based on the second-generation CAR structure to enhance the anti-tumor activity of CAR-T cells. For instance, CAR-T cells that secrete cytokines such as IL-12, IL-15, or IL-18 facilitate the proliferation and survival of CAR-T cells, thereby improving their anti-tumor activity.

Currently, it is believed that the reasons for the poor efficacy of CAR-T against solid tumors are as follows. It is associated with the complex environment and abnormal vascular structure in the solid tumors. Typically, leakiness or abnormal development of tumor vessels prevent cytotoxic T cells from entering the tumor. CAR-T cells lack expression of chemokines involved in infiltrating into tumor tissues, and dense physical barrier formed by tumor-associated fibroblasts (CAFs) outside the solid tumors and blood vessels limit CAR-T cell migration and infiltration into tumor tissues. Furthermore, the tumor microenvironment (TME) aids tumor cells in the evasion of immune surveillance, inhibits the body's anti-tumor response, and suppresses CAR-T cell infiltration, proliferation, and survival. The tumor microenvironment is one of the main reasons why CAR-T cells are ineffective against solid tumors. Therefore, designing and constructing a CAR-T cell capable of effectively infiltrating into tumor tissues and reversing the tumor's immunosuppressive microenvironment can potentially overcome the low response rates of existing immunotherapies, thereby exhibiting more effective anti-tumor effects.

In summary, the present invention constructs a novel type of CAR-T cells with high GM-CSF expression (CAR-GM-T) based on an improved second-generation CAR, aiming to mitigate the deficiencies and shortcomings present in the existing technology.

### SUMMARY

In view of this, the objective of the present invention is to provide a novel CAR expression vector and use thereof.

To achieve the above objective, the technical solution of the present invention is:

A CAR expression vector, comprising a polynucleotide encoding a chimeric antigen receptor (CAR) and a polynucleotide encoding an immunomodulatory factor, wherein the polynucleotide encoding the immunomodulatory factor is full length or fragment of a polynucleotide encoding a granulocyte-macrophage colony-stimulating factor (GM-CSF).

Further, the polynucleotide encoding the CAR is linked to the polynucleotide encoding the GM-CSF by a sequence encoding a self-cleaving peptide P2A.

Further, the CAR further comprises: an extracellular region for targeting an antigen target, wherein the antigen target is a tumor-specific antigen or a tumor-associated antigen; a transmembrane region for anchoring structure of the CAR; and an intracellular signal transduction domain comprising co-stimulatory molecules in tandem, the intracellular signal transduction domain comprises CD3ζ chain, 4-1BB, and GM-CSF.

Further, the antigen target is selected from the group consisting of Her2, B7-H3, Claudin18.2, CD70, MUC16, FSHR, FR, and Meso. Among these targets, targeting Her2 is mainly used for the treatment of HER2-positive breast cancer, lung cancer, gastric cancer, ovarian cancer, and sarcoma. Targeting Meso is used for the treatment of pancreatic cancer, ovarian cancer, and lung cancer. Targeting B7-H3 and CD70 are used for the treatment of melanoma. Targeting MUC16, FSHR, and FR are used for the treatment of ovarian cancer. Targeting Claudin18.2 is used for the treatment of gastric cancer, adenocarcinoma of the esophagogastric junction, and pancreatic cancer.

A lentivirus comprising the above CAR expression vector, wherein the lentivirus comprises pWPXLd, psPAX2, and/or pMD2.G.

A CAR-T cell, wherein the CAR-T cell expresses the above CAR expression vector.

Use of the above CAR-T cell in the preparation of a medicament for treating a solid tumor.

Preferably, the solid tumor mainly comprises breast cancer, ovarian cancer, and lung cancer, but are not limited to these.

Further, the CAR-T cell is capable of enhancing secretion of GM-CSF, IFN-γ, and IL-2.

Further, killing activity of the CAR-T cell is directly enhanced by expressing the GM-CSF.

Further, infiltration capability of the CAR-T cell is enhanced, and more of the CAR-T cells are capable of infiltrating into a solid tumor to exert effect of specifically killing a tumor cell.

Further, after infiltrating into the solid tumor, the CAR-T cell is capable of exerting an immune modulation function to regulate tumor microenvironment.

Further, the CAR-T cell is capable of activating and recruiting a dendritic cell internal of the solid tumor to trigger an antigen-specific tumor immune response of an endogenous T cell.

Further, the CAR-T cell is capable of inhibiting lymph node metastasis of a tumor cell.

The term “chemotaxis” in the present invention refers to a directed movement of dendritic cells toward tumor cells induced by the CAR-T cells (i.e., CAR-GM-T) prepared by the present invention.

Dendritic cells are specialized antigen-presenting cells that express MHC II and co-stimulatory molecules in the body. They can uptake, capture, and process tumor antigens.

Subsequently, they migrate to lymph nodes to activate the endogenous T cell anti-tumor immune response.

### Beneficial Effects

To further improve the therapeutic effect of CAR-T on solid tumors, the present invention constructs a CAR-T cell with high GM-CSF expression (CAR-GM-T) based on the second-generation CAR-T. Granulocyte-macrophage colony-stimulating factor (GM-CSF) is an important immune regulator. GM-CSF can activate and recruit dendritic cells (DC), trigger T-cell immune responses, and activate other immune cells such as granulocytes, macrophages, and NK cells, which play a crucial role in the regulation of the anti-tumor immune response. Experimental results show that CAR-GM-T cells constructed by the present invention are capable of expressing high-level GM-CSF, not only directly enhancing the killing activity and proliferation capacity of CAR-GM-T cells per se but also conferring these CAR-GM-T cells with stronger immune modulation functions than conventional CAR-T cells. Specifically, CAR-GM-T cells prepared by the present invention have outstanding effects in the following aspects:
1) CAR-GM-T cells have significantly enhanced killing of ovarian cancer cells compared to conventional CAR-T cells.
2) CAR-GM-T cells have significantly enhanced killing of ovarian cancer cells compared to Meso-CAR-T cells.
3) CAR-GM-T cells can significantly inhibit the growth of melanoma and significantly extend the survival time of experimental animals.
4) CAR-GM-T cells secrete immunomodulatory factors GM-CSF, IFN-γ, and IL-2 at significantly higher levels before and after contacting tumor cells compared to conventional CAR-T cells.
5) CAR-GM-T cells have a stronger capability to infiltrate into solid tumor tissues than conventional CAR-T cells and exert a stronger anti-tumor effect after infiltrating into solid tumors.
6) CAR-GM-T cells inhibit the lymph node metastasis of tumor cells.

In summary, the present invention can reshape and reverse the tumor microenvironment, and directly or auxiliarily enhance T cell activity, providing a new strategy for the treatment of solid tumors.

### DESCRIPTION OF DRAWINGS

To make the embodiments of the present invention or the technical solutions in the prior art clearer, the drawings required to be used in the description of the embodiments or the prior art will be briefly introduced below.

FIG. 1 illustrates the construction of the CAR-hGM expression vector.

FIG. 2 illustrates that CAR-GM-T cells secrete GM-CSF at significantly higher levels compared to conventional CAR-T cells.

FIG. 3 illustrates the killing activity of CAR-GM-T cells.

FIG. 4 illustrates that Meso-CAR-GM-T cells exhibit significantly higher killing efficiency against SK-OV3-Meso compared to Meso-CAR-T cells, with killing efficiency increasing as the effector-to-target ratio increases.

FIG. 5 illustrates the secretion of GM-CSF by CAR-GM-T cells during killing tumor cells is significantly higher than that by conventional CAR-T cells.

FIG. 6 illustrates that at an effector-to-target ratio of 5:1, CAR-GM-T cells secrete 4.5 times more IFN-γ than conventional CAR-T cells. The IFN-γ secreted by CAR-GM-T cells upon contact with tumor cells is significantly higher than that secreted by conventional CAR-T cells at different effector-to-target ratios.

FIG. 7 illustrates that CAR-GM-T cells exhibit significantly enhanced secretion of IL-2 when killing tumor cells at different effector-to-target ratios.

FIG. 8 illustrates the therapeutic effect of CAR-GM-T cells in a mouse intraperitoneal xenograft model.

FIG. 9 illustrates that conventional CAR-T cells have no significant inhibitory effect on the growth of B16F10-Her2 subcutaneous melanoma xenografts and do not prolong the survival of mice, while treatment with CAR-GM-T cells significantly inhibits the growth of subcutaneous melanoma xenografts of mice.

FIG. 10 illustrates that treatment with CAR-GM-T cells significantly prolongs the survival time of mice.

FIG. 11 illustrates that 24 days after treatment, mouse tumors and lymph nodes are isolated and prepared as single-cell suspensions, and the proportion of CAR-T cells in mouse tumors and lymph nodes is measured by flow cytometry.

FIG. 12 illustrates the further investigation of the infiltration of CAR-GM-T cells into human tumor tissues. 28 days after treatment in NSG mice with intraperitoneal xenografts, residual tumors in the peritoneal cavity are subjected to immunofluorescence staining to detect CD3⁺ T cell infiltration.

FIG. 13 illustrates that the proportion of CD45.2⁺ immune cells accounts for only 5% in the conventional CAR-T treatment group, and immune cell infiltration in mouse tumors in the CAR-GM-T treatment group is significantly higher compared to the conventional CAR-T treatment group.

FIG. 14 illustrates that CD3⁺ T lymphocytes account for 11% of CD45.2⁺ cells in the tumors of mice in the CAR-GM-T treatment group, whereas CD3⁺ T lymphocytes account for only 5% in the conventional CAR-T treatment group, suggesting that high expression of GM-CSF significantly increases the proportion of endogenous CD3⁺ T cells in the tumors of mice after CAR-T cell treatment.

FIG. 15 illustrates that the proportion of CD11c⁺MHCII^{hi} DC cells in the tumors of mice in the CAR-GM-T treatment group is significantly higher compared to the conventional CAR-T treatment group.

FIG. 16 illustrates the HE staining of tumor-draining lymph nodes showing that CAR-GM-T cells can inhibit lymph node metastasis of tumor cells.

### DETAILED DESCRIPTION

To make the objective, the technical solutions and advantages of the embodiments of the present invention clearer, the technical solutions of the embodiments of the present invention will be clearly and completely described below in combination with drawings. It is obvious that the described embodiments are some of the embodiments of the present invention, not all of the embodiments. Based on the embodiments of the present invention, all other embodiments obtained by those of ordinary skill in the art without making inventive effort shall belong to the protection scope of the present invention.

It should be noted that the term “include”, “comprise” or any variant thereof is intended to encompass nonexclusive inclusion so that a process, method, article or device including a series of elements includes not only those elements but also other elements which have been not listed definitely or an element(s) inherent to the process, method, article or device. Moreover, the expression “comprising a(n) . . . ” in which an element is defined will not preclude presence of an additional identical element(s) in a process, method, article or device comprising the defined element(s) unless further defined.

As used herein, the term “about”, typically means +/−5% of the stated value, more typically +/−4% of the stated value, more typically +/−3% of the stated value, more typically, +/−2% of the stated value, even more typically +/−1% of the stated value, and even more typically +1-0.5% of the stated value.

Throughout this application, embodiments of this invention may be presented with reference to a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc.; as well as individual numbers within that range, for example, 1, 2, 3, 4, 5, and 6. This applies regardless of the breadth of the range.

### Embodiment 1

### 1. Cell Culture

Human ovarian cancer cell line expressing Luciferase (SK-OV3-Luc) and mouse melanoma cell line overexpressing Her2 protein (B16F10-Her2), preserved by the State Key Laboratory of Biotherapy, Sichuan University, are cultured in DMEM medium containing 10% fetal bovine serum under normoxic culturing conditions at 37° C., 5% CO₂.

### 2. Preparation of CAR-GM-T

### 2.1 Construction and Validation of CAR-hGM Expression Vector

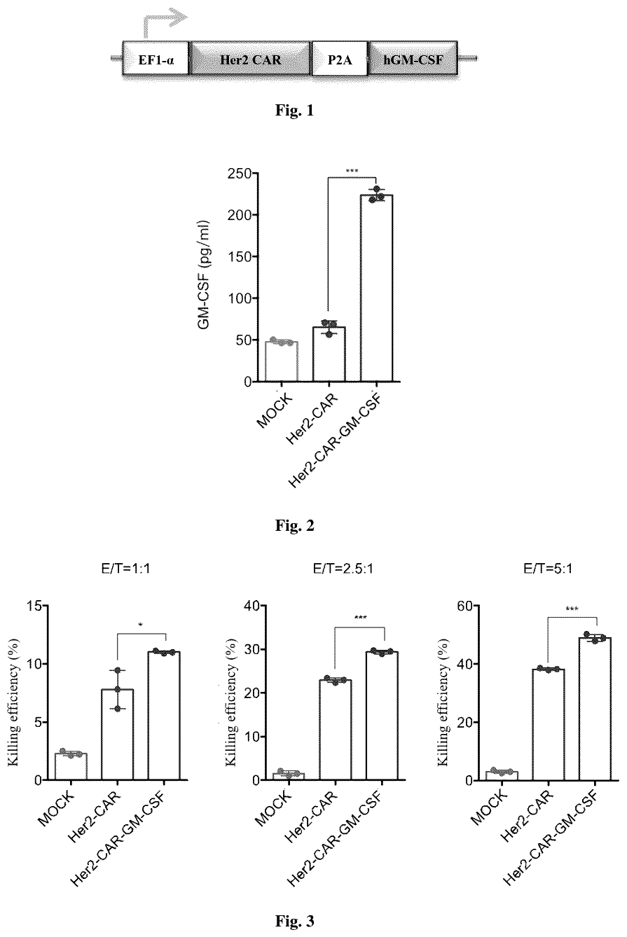

The Her2-CAR-hGM expression vector that expresses high-level hGM-CSF (pWPXLd-Her2-CAR-hGM-CSF) is constructed by linking Her2-CAR and hGM-CSF with a self-cleaving peptide P2A (FIG. 1). The Her2-CAR-P2A fragment is amplified using the pWPXLd-Her2-CAR-EGFP vector as a template, and the hGM-CSF fragment is amplified using the pWPXLd-hGM-CSF vector as a template. The vector pWPXLd is linearized using restriction endonucleases BamHI and EcoRI. The fragments Her2-CAR and hGM-CSF are ligated into the linearized vector pWPXLd by homologous recombination.

### 2.2 Packaging of Lentivirus

Lentiviral packaging is performed using a three-plasmid lentivirus production system.

Plasmid transfection is performed by the calcium phosphate-DNA co-precipitation method. Helper plasmids are psPAX2 and pMD2.G. Packaging cells are 293T cells. Viral supernatants are collected at 48 and 72 hours post-transfection respectively, filtered using a 0.22-μm disposable syringe filter (PES membrane), concentrated by ultracentrifugation, and then aliquoted and stored at −80° C. freezer.

### 2.3 Activation and Infection of Human T Cells

1) Peripheral blood mononuclear lymphocytes are isolated using Ficoll.
2) According to the number of peripheral blood mononuclear lymphocytes, an appropriate amount of Dynabeads Human T-Expander CD3/CD28 are washed once with 5 mL DPBS and resuspended in T cell complete culture medium. The peripheral blood mononuclear lymphocyte suspension is mixed with the washed Dynabeads and incubated in a T75 flask at 37° C. and 5% CO₂.
3) The virus dose is calculated according to the virus titer. RetroNectin is used to enhance the infection efficiency. CAR expression in each group (including the conventional CAR) is measured using flow cytometry 72 hours after infection.

### 2.4 Activation and Infection of Mouse T Cells

1) Lymph node cells are isolated from mouse lymph nodes.
2) According to the number of peripheral blood mononuclear lymphocytes, an appropriate amount of Dynabeads Human T-Expander CD3/CD28 are washed once with 5 mL DPBS and resuspended in T cell complete culture medium. The peripheral blood mononuclear lymphocyte suspension is mixed with the washed Dynabeads and incubated in a T75 flask at 37° C. and 5% CO₂.
3) The virus dose is calculated according to the virus titer. Polybrene is used to enhance the infection efficiency. CAR expression in each group (including the conventional CAR) is measured using flow cytometry 72 hours after infection.

### 2.5 CAR-GM-T Cell Culture

### 3. Cytokine Detection

The secretion of cytokines, including IL-2, IFNγ, and GM-CSF, is determined by ELISA. Samples are obtained from the supernatants of CAR-T cell cultures or the supernatants of cultures after CAR-T cell-induced killing of tumor cells.

### 4. Killing Activity Detection

In vitro killing activity is measured using a lactate dehydrogenase (LDH) cytotoxicity assay kit with specific steps described as follows:
1) Tumor cells in good growth condition are trypsinized, counted, and resuspended in X-VIVO medium to a final concentration of 5×10⁵ cells/mL.
2) CAR-T cells in good growth condition are gently dispersed, counted, and centrifuged at 1000 rpm for 3 minutes. The supernatant is discarded, and the pellet is resuspended in X-VIVO medium to a final concentration of 10⁶ cells/mL.
3) CAR-T cells are co-cultured with tumor cells at different effector-to-target ratios (1:1, 2.5:1, 5:1, 10:1), with tumor cells calculated as 10⁴ per well. Each effector-to-target ratio is set into the following six groups: MOCK T (spontaneous effector cell group), MOCK T+ Tumor (experimental group), CAR-T, CAR-T+ Tumor, CAR-GM-CSF-T, CAR-GM-CSF-T+ Tumor. Each group consists of 3 replicates, along with a blank culture medium group, a spontaneous tumor cell group, and a maximum tumor release group. The final volume per well is 200 μL.
4) 24 hours after killing, CAR-T cell-induced killing in each group is observed by microscope.
5) 20 μL of Lysis Solution (10×) is added to each well of the maximum tumor release group in the 96-well cell culture plate and incubated at 37° C. for 45 minutes.
6) After incubation, centrifugation is performed using a horizontal centrifuge at 250 g for 4 minutes at room temperature.
7) 50 μL of supernatant from each well is transferred to a new 96-well plate for subsequent LDH release assay.
8) 50 μL of LDH reaction solution is added to each well and incubated in the dark at room temperature for 30 minutes.
9) After incubation, 50p of stop solution is added to each well.
10) After the reaction is stopped, the absorbance at 490 nm is read using a microplate reader within 1 hour.

$$11 )$$

$$Killing efficiency \% = \frac{Experimental group - Spontaneous effector cell group - Spontaneous tumor cell group + Blank culture medium}{Maximum tumor release group - Spontaneous tumor cell group} \times 100$$

### 5. Treatment of Intraperitoneal Xenograft Model of Ovarian Cancer (an Ovarian Cancer Model Established by Intraperitoneal Injection of Ovarian Cancer Cells)

### 5.1 Animal Maintenance

1) The animals used for this section of the experiment are B-NDG® mice (NOD-Prkdcscid Il2rgtml/Bcgen) generated by Biocytogen. These mice are on the NOD-scid genetic background with IL2rg gene knockout and lack T, B, and NK cells, making them suitable for human-derived cell or tissue engraftment.
2) Purchased 5-6-week-old female B-NDG mice, weighing 18-20 g, are kept in the SPF-level animal facility of State Key Laboratory of Biotherapy, Sichuan University.
3) During the maintenance of B-NDG mice, the room temperature is maintained at 25° C. The mice have ad libitum access to food and water during the experiment.

### 5.2 Construction of Intraperitoneal Xenograft Model of Ovarian Cancer

1) SK-OV3-Luc cells in good growth condition are trypsinized, counted, centrifuged at 1200 rpm for 3 minutes, washed twice with PBS, and resuspended in serum-free DMEM medium to a final concentration of 10⁶ cells/ml.
2) Each mouse is grabbed and inoculated with 200 μL of SK-OV3-Luc cell suspension by intraperitoneal injection.
3) The body weights of mice are monitored regularly.
   5.3 Treatment of intraperitoneal xenograft model of ovarian cancer
1) On the 6th day after inoculation, each mouse is subjected to in vivo imaging. Based on the fluorescence value of the image, the mice are divided into 3 groups with 6 mice per group: PBS group, Her2-CAR group, Her2-CAR-GM-CSF group.
2) On the 7th day after inoculation, Her2-CAR-T and Her2-CAR-GM-CSF-T cells in good growth condition with a CAR positive rate of over 50% are collected and resuspended in X-VIVO medium to a final concentration of 10⁷ cells/mL.
3) 100 μL of the above CAR-T cell suspension is aspirated and treated to each mouse by intraperitoneal injection.

### 5.4 Detection of Therapeutic Efficacy in Intraperitoneal Xenograft Model of Ovarian Cancer

1) After the mice are subject to the treatment, in vivo imaging is performed once a week.
2) The mice are being regularly monitored for their mental status, activity levels, fur glossiness, eating conditions, as well as for the occurrence of adverse reactions. The body weights of the mice are measured.
3) After monitoring, the mice are euthanized via cervical dislocation under anesthesia. The residual tumors in the peritoneal cavity are collected for immunofluorescence staining to assess the infiltration of CAR-T cells.

### 6. Treatment of Subcutaneous Melanoma Xenograft Model

### 6.1 Animal Maintenance

(1) C57BL/6 CD45.1 mice preserved by our laboratory and C57BL/6 CD45.2 mice purchased from Sichuan University's Laboratory Animal Center are used for this animal experiment.
(2) Purchased 5-6-week-old female C57BL/6 CD45.2 mice, weighing 18-20 g, are kept in the SPF-level animal facility of Sichuan University.
(3) During the maintenance of the mice, the room temperature is maintained at 25° C. The mice have ad libitum access to food and water.
   6.2 Construction of subcutaneous melanoma xenograft model overexpressing Her2 protein
(1) The model is constructed using C57BL/6 CD45.2 mice. The mice are used for the construction of subcutaneous melanoma xenograft model one week following their purchase.
(2) B16F10-Her2 cells in good growth condition are trypsinized, counted, centrifuged at 1200 rpm for 3 minutes, washed twice with PBS, and resuspended in serum-free DMEM medium to a final concentration of 5×10⁶ cells/ml.
(3) 100 μL of the above tumor cell suspension is injected subcutaneously into the right thorax of the mice. Be careful not to penetrate the muscular layer.

### 6.3 Treatment of Subcutaneous Melanoma Xenograft Model

(1) 200 mg of Cyclophosphamide (CPA) is dissolved in 5 mL of normal saline, filtered using a 0.22-μm disposable syringe filter, and then aliquoted and stored at −20° C. freezer.
(2) On the 10th day after inoculation, each tumor-bearing mouse is injected intraperitoneally with a dose of 200 mg/kg of cyclophosphamide.
(3) On the 12th day after inoculation, the xenograft volume is measured. Based on the tumor volume, mice are divided into 3 groups with 15 mice per group: PBS group, Her2-CAR group, Her2-CAR-GM-CSF group.
(4) On the 13th day after inoculation, Her2-CAR-T and Her2-CAR-GM-CSF-T cells in good growth condition prepared by C57BL/6 CD45.1 mouse lymphocytes are collected and resuspended in 1640 medium to a final concentration of 3×10⁷ cells/mL.
(5) 100 μL of the above CAR-T cell suspension is aspirated and treated to each mouse by tail vein injection.

Detection of therapeutic efficacy in subcutaneous melanoma xenograft model
(1) Tumor volume measurements of the mice are conducted every 3 days. The longitudinal and transverse diameters of tumors are measured using a Vernier caliper and recorded. The longest and shortest diameters of subcutaneous melanoma xenografts are measured. Tumor volume is calculated using the formula: Tumor Volume=0.52×length×width×width. The tumor growth curve is plotted.
(2) The mice are being regularly monitored for their mental status, activity levels, fur glossiness, eating conditions, as well as for the occurrence of adverse reactions. The body weights of the mice are measured. The survival curve is plotted.
(3) 21 days after treatment, some mice are euthanized via cervical dislocation under anesthesia. The spleens, tumor-draining lymph nodes, lymph nodes, and tumors are isolated for Hematoxylin and Eosin (HE) staining and flow cytometric analysis, respectively.

### Embodiment 2

### Experimental Results

### 1. Enhanced GM-CSF Secretion Capability of CAR-GM-T Cells

There is no significant difference in GM-CSF secretion between conventional CAR-T cells and MOCK T cells before activation. However, the GM-CSF secretion by CAR-GM-T cells is significantly higher than that of conventional CAR-T cells (P<0.001), as expected (FIG. 2).

### 2. Enhanced Killing Activity of CAR-GM-T Cells

The killing efficiency of Her2-CAR-GM-T cells against SK-OV3 cells naturally expressing Her2 protein is significantly higher than that of Her2-CAR-T cells, and the killing efficiency increases as the effector-to-target ratio increases. The killing efficiency of Her2-CAR-GM-T cells against SK-OV3 reaches 30% at an effector-to-target ratio of 1:1, significantly higher than that of Her2-CAR-T cells (P<0.001). In contrast, the killing efficiency of Her2-CAR-T cells reaches 30% at an effector-to-target ratio of 2.5:1. The killing efficiency of Her2-CAR-GM-T cells is close to 80% at an effector-to-target ratio of 5:1, significantly higher than the 60% of killing efficiency of Her2-CAR-T cells of (FIG. 3).

Detection of the killing efficiency of CAR-GM-T cells targeting Meso (Meso-CAR-GM-T) on Meso-positive tumor cells.

Meso CAR-T is a CAR-T therapy targeting Meso developed by the University of Pennsylvania and Novartis. Meso CAR-T is also the first CAR-T product launched by Novartis for solid tumors, with indications including ovarian cancer, lung cancer, and pancreatic cancer.

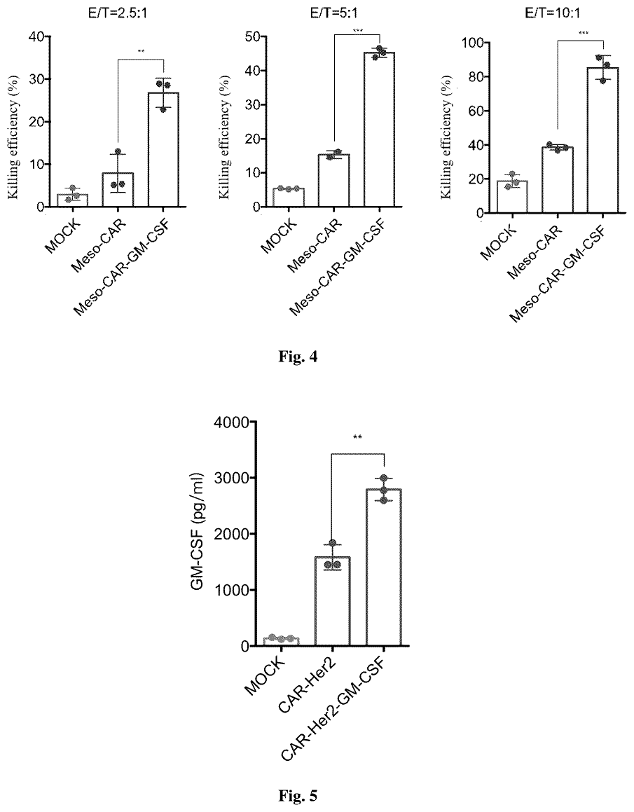

The results show that Meso-CAR-GM-T cells exhibit a significantly higher killing efficiency against SK-OV3-Meso compared to Meso-CAR-T cells, and the killing efficiency increases as the effector-to-target ratio increase (FIG. 4). These results indicate that the high expression of CAR-GM-CSF is effective not only against the Meso target but also enhances the in vitro killing efficiency of CAR-T cells against different targets.

3. Enhanced Secretion of GM-CSF, IFN-γ, and IL-2 by CAR-GM-T Cells during Killing Tumor Cells

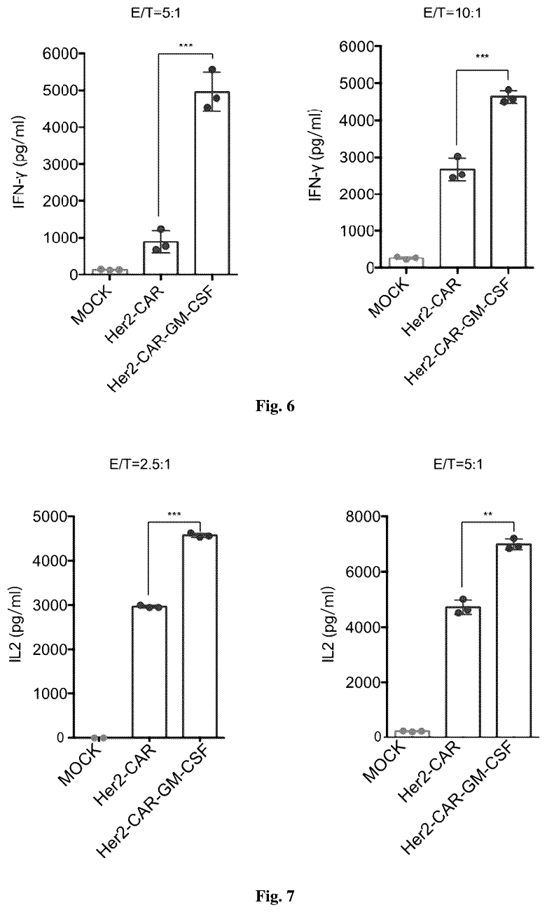

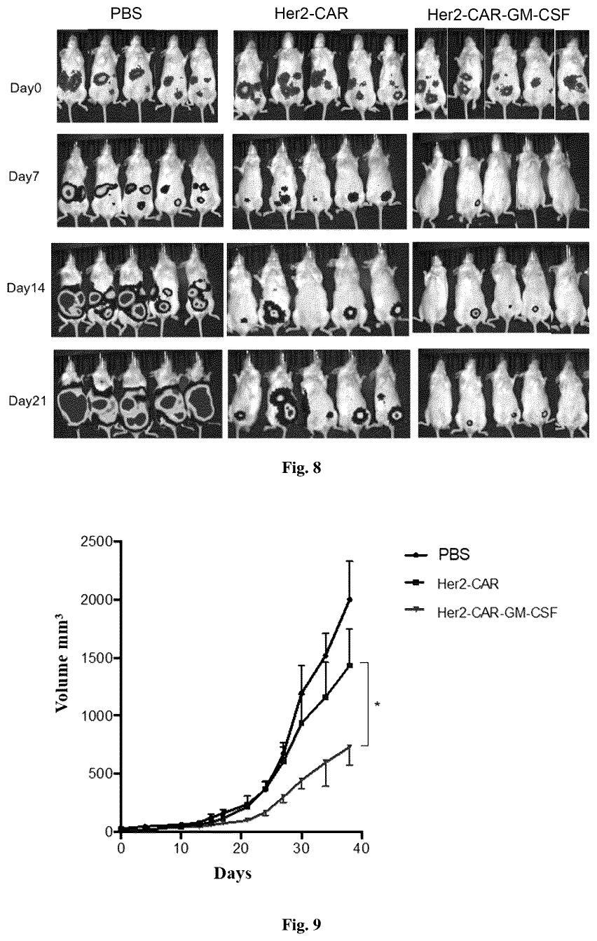

The secretion of cytokines in the supernatant when CAR-T cells kill tumor cells is examined. After activation upon contact with tumor cells, CAR-GM-T cells show an enhanced capability to secrete GM-CSF compared to the conventional CAR-T cells (P<0.01) (FIG. 5). At an effector-to-target ratio of 5:1, CAR-GM-T cells secreted 4.5 times more IFN-γ than CAR-T cells, and the secretion of IFN-γ by CAR-GM-T cells is significantly higher than that of CAR-T cells at different effector-to-target ratios (P<0.001) (FIG. 6). Furthermore, at different effector-to-target ratios, CAR-GM-T cells show significantly enhanced secretion of IL-2 during killing tumor cells (FIG. 7).

### 4. Enhanced Therapeutic Effect of CAR-GM-T Cells in Ovarian Cancer

Conventional CAR-T cells could control the tumor growth of the mice, with one mouse showing tumor elimination on the 14th day of treatment, but the tumor recurs on the 21st day. In contrast, on the 14th day of treatment with CAR-GM-T cells, two mice exhibit complete tumor regression, and one mouse shows substantial tumor regression. On the 21st day, among the mice with tumor regression, 2 mice have no tumor recurrence, and the remaining 3 mice have very small tumor loads that are significantly smaller than those of mice treated with conventional CAR-T cells. These results indicate that CAR-GM-T cells have a more significant in vivo anti-tumor effect than conventional CAR-T cells.

### 5. Enhanced Effect of CAR-GM-T Cells on High HER2-Expressing Subcutaneous Melanoma Xenografts in Immunocompetent Mice

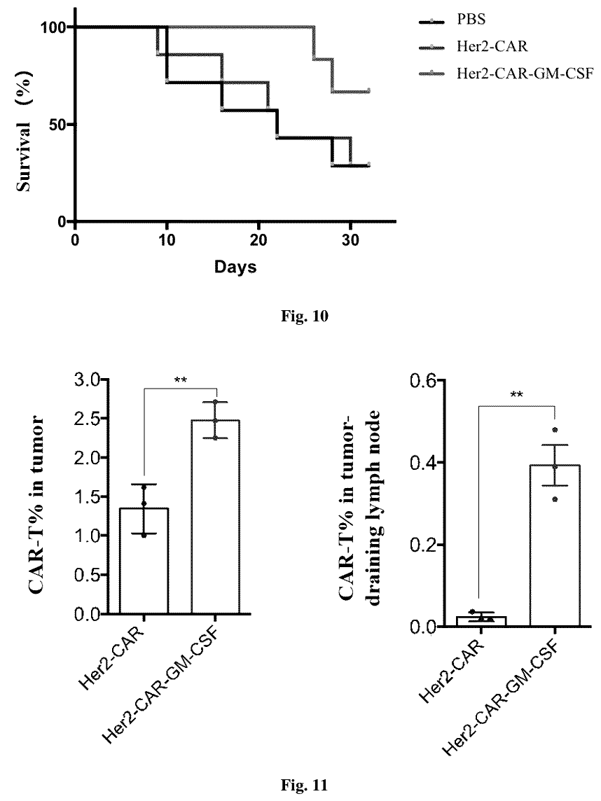

CAR-T cells show no significant inhibitory effect on the growth of B16F10-Her2 melanoma subcutaneous xenografts and do not extend the survival of mice. In contrast, treatment with CAR-GM-T cells significantly inhibits the growth of melanoma subcutaneous xenografts in mice (P<0.05) (FIG. 9), leading to a substantial extension in the survival time of mice (FIG. 10).

0.6. Enhanced Infiltration of CAR-GM-T Cells into Solid Tumors

On the 24th day after treatment, tumors, and lymph nodes are isolated and prepared into single-cell suspensions. Flow cytometry is used to detect the proportion of CAR⁺ T cells in CD3⁺ T cells. The results show that in the conventional CAR-T treatment group, CAR⁺ T cells in the tumor account for 1.2% of CD3⁺ T cells. In the CAR-GM-T treatment group, CAR⁺ T cells in the tumor account for 2.4%, which is twice that of the conventional CAR-T treatment group and statistically significant (P<0.01). Similar results are obtained by analyzing the proportion of CAR⁺ T cells in tumor-draining lymph nodes. In the conventional CAR-T treatment group, CAR⁺ T cells in tumor-draining lymph nodes account for only 0.04% of CD3⁺ T cells. In the CAR-GM-T treatment group, CAR⁺ T cells in tumor-draining lymph nodes account for 0.4%, which is 10 times higher than that in the conventional CAR-T treatment group and significantly higher (P<0.01) (FIG. 11).

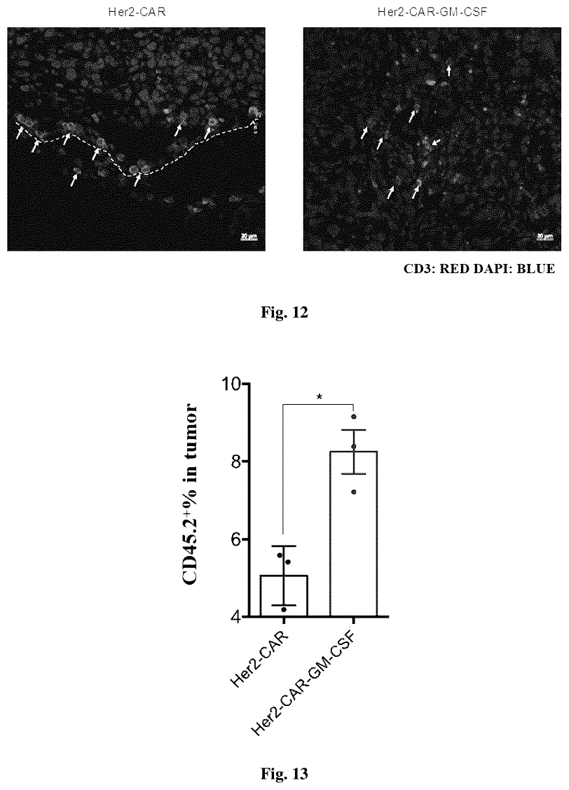

The infiltration of CAR-GM-T cells into human tumor tissues is further investigated. 28th day after the treatment of intraperitoneal xenografts in NSG mice, the residual tumors in the peritoneal cavity are subjected to immunofluorescence staining to detect the infiltration of human-derived CD3⁺ T cells. The results show that in mice treated with CAR-T, human-derived CD3⁺ T cells mainly aggregate at the edge of the tumor, with few T cells inside the tumor. In contrast, in mice treated with CAR-GM-T cells, human-derived CD3⁺ T cells could infiltrate into the tumor, and the number of CD3⁺ T cells in the internal tumor tissues is significantly increased, indicating that high expression of GM-CSF not only enhances the killing activity of CAR-T cells but also increases the capability of CAR-T cells to infiltrate into the tumor (FIG. 12). These results indicate that high expression of GM-CSF enhances the infiltration capability of CAR-T cells, and CAR-GM-T cells can more effectively enter tumors and lymph nodes to exert their functions.

### 7. Activation of Endogenous T Cells by CAR-GM-T Cells

Analysis of CD45.2⁺ endogenous immune cells in mouse tumor cells is performed to investigate the infiltration of endogenous immune cells. The results show that in the CAR-GM-T treatment group, CD45.2⁺ immune cells in the mouse tumors account for 8%, while in the CAR-T treatment group, CD45.2⁺ immune cells account for only 5%. The infiltration of immune cells into mouse tumors in the CAR-GM-T treatment group is significantly better than that in the CAR-T treatment group (P<0.05) (FIG. 13).

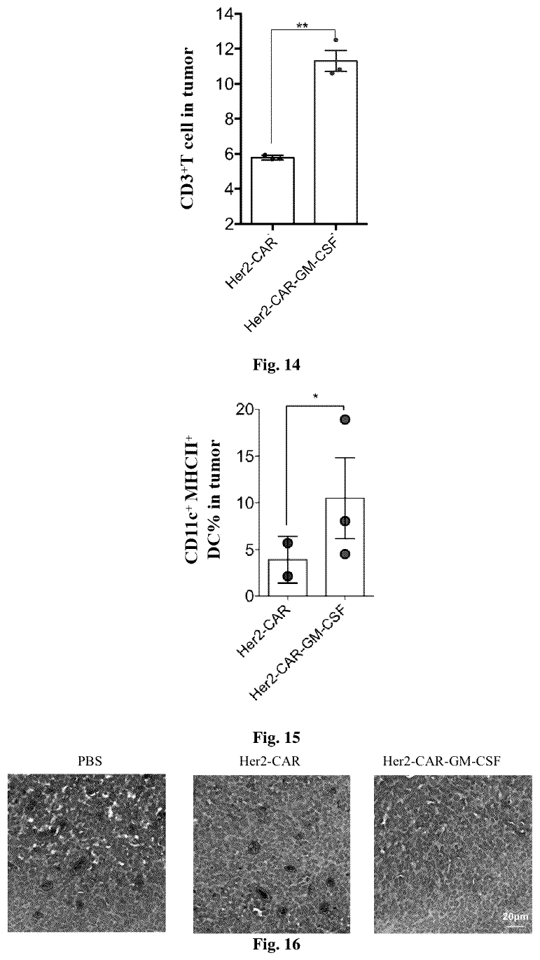

Analysis of CD3⁺ T lymphocytes in CD45.2⁺ endogenous immune cells shows that in the CAR-GM-T treatment group, CD3⁺ T lymphocytes in mouse tumors account for 11% of CD45.2⁺ cells. While in the CAR-T treatment group, CD3⁺ T lymphocytes account for only 5%. This indicates that high expression of GM-CSF significantly increases the proportion of endogenous CD3⁺ T cells in mouse tumors after CAR-T cell treatment (P<0.01) (FIG. 14).

8. Chemotaxis of Dendritic Cells (DCs) toward Tumors Induced by CAR-GM-T Cells

Analysis of CD11b⁺CD11c⁺ cells in CD45.2⁺ cells is performed, followed by the analysis of CD11c⁺MHCII^{hi} DCs in the population of CD11b⁺CD11c cells. Results show that in the CAR-GM-T treatment group, the proportion of CD11c⁺MHCII^{hi} DCs in the tumors is significantly higher than those in the conventional CAR-T treatment group (P<0.01) (FIG. 15), indicating that there are more mature DCs internal of the tumors in the CAR-GM-T treatment group.

### 9. Inhibition of Lymph Node Metastasis by CAR-GM-T Cells

B16F10 is a murine melanoma cell line with higher metastatic potential. The research team of the present invention uses CAR-T cells and CAR-GM-T cells to treat the B16F10-Her2 melanoma subcutaneous xenograft model. Hematoxylin and eosin (HE) staining of tumor-draining lymph nodes shows that the PBS treatment group has a large number of melanin spots, the CAR-T treatment group has a small number of melanin spots, and the CAR-GM-T treatment group has a large number of lymphocytes with no melanin spots. These results indicate that in mice without treatment, there are a large number of tumor cells metastasizing to the lymph nodes. CAR-T treatment could inhibit the metastasis of some tumor cells, and high expression of GM-CSF enhances the capability of CAR-T cells to inhibit the lymph node metastasis of tumor cells (FIG. 16).

The embodiments of the present invention are described above with reference to the accompanying drawings, but the present invention is not limited to the aforementioned specific embodiments. The aforementioned embodiments are merely illustrative and not limiting. For those of ordinary skill in the art, many forms can be made under the teaching of present invention without departing from the spirit of the present invention and the scope of the claims, all of which shall fall within the protection scope of the present invention.

1. A CAR-T cell, wherein, the CAR-T cell expresses a chimeric antigen receptor (CAR) and an immunomodulatory factor via viral transduction; the polynucleotide encoding the immunomodulatory factor is full length of a polynucleotide encoding a granulocyte-macrophage colony-stimulating factor (GM-CSF), wherein the CAR further comprises: an extracellular region for targeting an antigen target, wherein the antigen target is a tumor-specific antigen or a tumor-associated antigen; a transmembrane region for anchoring structure of the CAR; and an intracellular signal transduction domain comprising co-stimulatory molecules in tandem, the intracellular signal transduction domain comprises CD3ζ chain and 4-1 BB.

2. The CAR-T cell according to claim 1, wherein the polynucleotide encoding the CAR is linked to the polynucleotide encoding the GM-CSF by a sequence encoding a self-cleaving peptide P2A.

3. (canceled)

4. The CAR-T cell according to claim 1, wherein the antigen target is selected from the group consisting of Her2, B7-H3, Claudin18.2, CD70, MUC16, FSHR, FR, and Meso.

5. A lentivirus vector comprising the CAR expression vector, wherein the lentivirus vector comprises a polynucleotide encoding a chimeric antigen receptor (CAR) and a polynucleotide encoding an immunomodulatory factor; wherein the polynucleotide encoding the immunomodulatory factor is full length of a polynucleotide encoding a granulocyte-macrophage colony-stimulating factor (GM-CSF); wherein the CAR comprises: an extracellular region for targeting an antigen target, wherein the antigen target is a tumor-specific antigen or a tumor-associated antigen; a transmembrane region for anchoring structure of the CAR; and an intracellular signal transduction domain comprising co-stimulatory molecules in tandem, the intracellular signal transduction domain comprises CD3ζ chain and 4-1 BB; wherein the lentivirus comprises pWPXLd, psPAX2, and/or pMD2.G.

6. The lentivirus vector according to claim 4, wherein the polynucleotide encoding the CAR is linked to the polynucleotide encoding the GM-CSF by a sequence encoding a self-cleaving peptide P2A.

7-13. (canceled)

14. The lentivirus vector according to claim 4, wherein the antigen target is selected from the group consisting of Her2, B7-H3, Claudin18.2, CD70, MUC16, FSHR, FR, and Meso.

15. A method of treating a patient having a solid tumor, comprising:
selecting the patient having the solid tumor;
preparing a medicament comprising the CAR-T cell according to claim 1; and
administrating the medicament to the patient.

16. The method according to claim 15, wherein the solid tumor expresses an antigen target, and the antigen target is selected from the group consisting of Her2, B7-H3, Claudin18.2, CD70, MUC16, FSHR, FR, and Meso.

17. The method according to claim 15, wherein the solid tumor is selected from the group consisting of breast cancer, lung cancer, gastric cancer, ovarian cancer, sarcoma, pancreatic cancer, melanoma, and adenocarcinoma of the esophagogastric junction.

18. The method according to claim 15, wherein the solid tumor exhibits metastatic potential to the lymph nodes.

19. The method according to claim 15, wherein the step of administrating the medicament to the patient is by intraperitoneal injection.
